(19) United States
(12) Patent Application Publication
(10) Pub. No.: US 2010/0249639 A1
(43) Pub. Date: Sep. 30, 2010

(54) AIRWAY MANAGEMENT DEVICES, ENDOSCOPIC CONDUITS, SURGICAL KITS, AND METHODS OF USING THE SAME

(76) Inventor: Samir Bhatt, Wellesley, MA (US)

Correspondence Address: EDWARDS ANGELL PALMER & DODGE LLP, P.O. BOX 55874, BOSTON, MA, 02205, US

(21) Appl. No.: 12/705,592

(22) Filed: Feb. 13, 2010

Related U.S. Application Data
(60) Provisional application No. 61/211,312, filed on Mar. 26, 2009.
(60) Provisional application No. 61/212,435, filed on Apr. 11, 2009.

(51) Int. Cl. A61M 16/04; A61B 1/04; A61B 5/0488

(52) U.S. Cl. 600/546; 128/207.14; 600/120; 600/109; 600/101

(57) ABSTRACT

One aspect of the invention provides an airway management device including: an endotracheal tube having a proximal end and a distal end and a sheath adjacent to the endotracheal tube. The sheath includes a proximal end and a distal end. The distal end of the endotracheal tube extends beyond the distal end of the sheath. Another aspect of the invention provides a surgical kit including an airway management device and instructions for use. The airway management device includes: an endotracheal tube having a proximal end and a distal end and a sheath adjacent to the endotracheal tube. The sheath includes a proximal and a distal end. The distal end of the endotracheal tube extends beyond the distal end of the sheath.

### RELATED APPLICATIONS

This application claims priority to U.S. Provisional Patent Application Ser. No. 61/211,312, filed Mar. 26, 2009 and U.S. Provisional Patent Application Ser. No. 61/212,435, filed Apr. 11, 2009. The entire contents of each application are incorporated herein by reference.

### BACKGROUND

The thyroid gland is a highly vascular structure that is located in the neck. It produces hormones that have widespread effects in the body. Removal of this gland, called thyroidectomy, is a commonly performed operation. Thyroidectomy is performed for many reasons, including diagnosis and treatment of tumors, and for control of an over- or under-functioning gland. The technique of thyroidectomy involves division of the soft tissue attachments that hold the gland in place.

The thyroid's location in the neck places numerous important structures in jeopardy during thyroidectomy. The recurrent laryngeal nerve (often abbreviated as RLN) is one of these vital structures at risk during thyroidectomy. This nerve controls motion of the arytenoids cartilages and vocal cords. There is one RLN located on each side of the neck. If this nerve is injured on one side of the neck during the surgery, the patient's voice can be severely impaired, often permanently, and the patient is at increased risk of developing pneumonia (“aspiration”). If the RLN is injured on both sides of the neck, the patient's airway can be impaired, thereby jeopardizing respiration.

The RLN is a long thin structure, only about 1.5 mm wide, that ascends from the chest into the neck where it enters the larynx (voice box) at the cricothyroid joint about 2 cm below the thyroid notch (“Adam's apple”). The nerve is located on the posterior (back) surface of the thyroid lobe on each side, and is usually within millimeters of the thyroid gland capsule and often abuts it directly. Sometimes the nerve is embedded within the gland or adherent to it, making dissection and preservation of the RLN even more technically difficult.

Proper dissection of the thyroid gland almost always requires dissection and division of soft tissue attachments within millimeters of the recurrent laryngeal nerve, and sometimes requires the surgeon to divide, manipulate, cauterize, and coagulate tissues directly on or adjacent to the nerve. Hence, the RLN is almost always at risk of injury during thyroid surgery. Similarly any other neck surgery occurring in this area (for example, parathyroid surgery, branchial cleft surgery, cervical esophageal surgery, cervical spine surgery and carotid artery surgery), will include dissection near the RLN, putting it at risk of injury. Clearly, patients benefit when the surgeon can accurately identify the recurrent laryngeal nerve during surgery and avoid injury to it during dissection of adjacent tissues. Surgeons take many different approaches to this objective.

Some surgeons avoid exposing the nerve, hoping that by staying at some distance from it, the likelihood of injury is reduced. This technique of avoiding exposure of the nerve has the frequent disadvantage of incomplete removal of diseased thyroid tissue, which can in some cases increase the risk of recurrent disease or cancer. Other surgeons try to identify the RLN at some point during surgery, and then dissect the thyroid gland away from this the nerve.

In all approaches, however, at some point during the operation a putative RLN may be encountered. It is then desirable to confirm that the structure encountered (the “nerve candidate”) is, in fact, the RLN as opposed to fascia, fat, blood vessels, or other tissues that may have a similar appearance. The unique characteristic that distinguishes the RLN from any other structure in the body is that, with electrical stimulation of the RLN, the muscles of the arytenoid and vocal cord on the same (ipsilateral) side of the larynx will twitch. This twitch in most cases cannot be visualized directly in the wound, because the muscles involved are on the posterior (back) side of the larynx, or internal to the larynx.

Instead, surgeons use other techniques to determine whether a nerve candidate is or is not the RLN. The most common techniques are listed below:

1.) The surgeon stimulates the nerve candidate electrically and monitors the laryngeal muscles by palpation for evidence of a muscle twitch. The palpation is done by placing a finger on the muscles on the external surface of the larynx, via the surgical field.

2.) The surgeon stimulates the nerve candidate electrically and monitors the laryngeal muscles electrically for evidence of a muscle twitch by using electrodes. These electrodes are placed at the beginning of the operation, either external or internal to the larynx.

3.) The surgeon stimulates the nerve candidate electrically and monitors the laryngeal muscles visually for evidence of a muscle twitch. The surgeon or other observer looks for motion of the vocal cord through endoscopes passed to the larynx via the mouth. In general, such endoscopes are passed at points during the operation when the nerve is being tested, then removed, allowing only intermittent monitoring of the nerve.

All of these techniques as currently used have drawbacks that are avoided and/or overcome by the invention.

### SUMMARY OF THE INVENTION

The present invention provides advantages over currently used surgical devices and techniques. One advantage pertains to identification and preservation of the recurrent laryngeal nerve during neck surgery. Another advantage relates to airway control and passage of endotracheal tubes. These and other advantages of the device are described below.

During thyroid surgery, the surgeon must thoroughly remove the thyroid gland without traumatizing the RLN, thereby maintaining normal vocal cord function. Embodiments of the invention permit identification and monitoring of recurrent laryngeal nerve (RLN) function during neck surgery. Embodiments of the invention allow the surgeon to continuously monitor the vocal cord and/or arytenoids visually, looking for twitches in response to electrical stimulation of the RLN during neck dissection and at the end of surgery prior to removal of the patient's airway. These techniques offer benefits over other approaches including a low incidence of false negative and false positive identification rates of RLN function, continuous monitoring of the RLN and airway, and ease of use. The devices provided by the invention enable a surgeon to perform thyroid and other neck surgery with greater confidence, thoroughness, and speed and with decreased risk to the RLN.

It is also important that the surgeon and anesthetist or anesthesiologist know the status of vocal cord function prior to, and at the conclusion of, surgery. The devices provided by the invention permit visualization of the vocal cords during patient spontaneous respiration. During spontaneous respiration the vocal cords abduct (move apart) to allow more air to pass. An arytenoid and/or vocal cord that is impaired in its motion by disease or tumor, or the RLN of which is paralyzed for any reason (such as by cancer, or that has been surgically injured) will be immobile during spontaneous inspiration. By contrast, the opposite normal vocal cord will move normally.

By using the devices provided by the invention, an anesthetist or anesthesiologist and surgeon can confirm prior to incision that the patient's vocal cords and RLN have intact function, or if it is abnormal, to assess the abnormality in detail. They can also confirm at the conclusion of surgery that the vocal cords and RLN function normally, or that they do not function normally, and make an appropriate response.

As mentioned above, there are three major methods of RLN monitoring currently in use. The first two methods generally employ an endotracheal tube and the third method generally uses a laryngeal mask airway. Some patients are not amenable to the third technique, such as those with large compressive thyroid masses. These patients often need a rigid endotracheal tube to stent the trachea in the region of the large thyroid mass, preventing compression and obstruction by the mass. This need may not always be apparent at the beginning of surgery, but may become more obvious over time, or with swelling or bleeding of the mass, or pressure from dissection.

In such cases, the surgeon or anesthesiologist may decide to “convert” from a laryngeal mask airway to an endotracheal tube at some point during the procedure. If these patients are anesthetized using a laryngeal mask airway alone, and subsequently need an endotracheal tube, the process of switching from the laryngeal mask to the endotracheal tube can be time consuming and cumbersome. This can be hazardous to the patient as he/she is not receiving ventilation (or anesthesia) during the conversion from one airway to another. The devices of the invention permit visual monitoring of the vocal cord as is done using this laryngeal mask airway technique while using an endotracheal tube that will stent the airway. No removal or conversion of airway is necessary and a stable, satisfactory airway is retained throughout the procedure.

Another advantage of embodiments of the invention is that they permit passage of an endotracheal tube under direct visualization of the vocal cords. The anesthetist or anesthesiologist is able to pass the endotracheal tube with better control than would occur without adequate vocal cord visualization, thereby reducing the risk of laryngeal injury or loss of airway control that can occur from incorrect or traumatic endotracheal tube passage. This advantage applies not only to surgeries that involve the neck but any induction of general anesthesia using an endotracheal tube for any reason. The improved view provided by embodiments of the invention allows more precise, safer airway control and intubation with an endotracheal tube than using a typical anesthesia laryngoscope.

The devices of the invention enable airway management with less disruption in a system that also allows continuous nerve monitoring during neck surgery.

Various exemplary aspects and embodiments of the invention are summarized below and described in greater detail herein.

One aspect of the invention provides an airway management device including: an endotracheal tube having a proximal end and a distal end and a sheath adjacent to the endotracheal tube. The sheath includes a proximal end and a distal end. The distal end of the endotracheal tube extends beyond the distal end of the sheath.

This aspect can have a variety of embodiments. In one embodiment, the airway management device includes a connector coupled to the endotracheal tube. The connector is adapted to interface with an anesthetic breathing circuit. The connector can be removably coupled to the endotracheal tube. The connector can be tethered to the endotracheal tube.

The endotracheal tube can be flexible, rigid, and/or armored. The sheath can be coupled to the endotracheal tube. The sheath can be coupled externally to the endotracheal tube. The sheath can be coupled internally to the endotracheal tube. The sheath can be confluent with the endotracheal tube. The sheath can be positioned on a concave (superior) side of the endotracheal tube. The endotracheal tube can include metal inserts to adjust and stiffen the endotracheal tube. The sheath can include one or more metal inserts to adjust and stiffen the endotracheal tube. The sheath can include one or more channels for transportation of fluids.

The airway management device can include one or more cuffs coupled to the sheath. The airway management device can include one or more inflation tubes coupled to the one or more cuffs. The one or more cuffs can be configured to dilate a subject's throat. The distal end of the sheath can extend beyond the one or more cuffs. The distal end of the sheath can extend between about 0 cm and about 10 cm beyond the one or more cuffs. The sheath can have an internal diameter selected from the group consisting of: less than about 2 mm, between about 2 mm and about 10 mm, and greater than about 10 mm. The distal end of the sheath can be configured to provide a view of a subject's vocal cords when the airway management device is placed in the larynx of the subject.

The airway management device can include a lens coupled to an opening at the distal end of the sheath. The airway management device can include a prism coupled to an opening at the distal end of the sheath.

The airway management device can include a cap coupled to an opening at the proximal end of the sheath. The cap can be tethered to the sheath.

The airway management device can include a flap coupled to the proximal opening of the sheath. The airway management device can include a flap coupled to the distal opening of the sheath.

The sheath can be configured to receive an endoscope. The airway management device can include an endoscope having a proximal end and a distal end. The airway management device can include a connector coupled to the distal end of the endotracheal tube and configured to immobilize the endoscope. The endoscope can include a light source. The endoscope can include a camera located at the distal end of the endoscope. The endoscope can include an ocular located at the proximal end of the endoscope. The endoscope can include a first fiber optic cable for transmission of light from the proximal end of the endoscope to the distal end of the endoscope. The endoscope can include a second fiber optic cable for transmission of images from the distal end of the endoscope to the proximal end of the endoscope. The endoscope can includes one or more channels for transportation of fluids. The endoscope can be rigid, semi-rigid, and/or flexible. The endoscope can be steerable.

The airway management device can include an additional cuff coupled to the endotracheal tube.

Another aspect of the invention provides a surgical kit including an airway management device and instructions for use. The airway management device includes: an endotracheal tube having a proximal end and a distal end and a sheath adjacent to the endotracheal tube. The sheath includes a proximal and a distal end. The distal end of the endotracheal tube extends beyond the distal end of the sheath.

This aspect of the invention can have a variety of embodiments. The endotracheal tube can be adapted for passage through a subject's vocal cords. The airway management device can include one or more cuffs coupled to the sheath. The surgical kit can include an endoscope.

Another aspect of the invention provides a method of identifying a subject's recurrent laryngeal nerve using the airway management devices of the invention as herein described in all its various aspects and embodiments. The method includes: introducing an airway management device into the subject's larynx, the airway management device including an endotracheal tube having a proximal end and a distal end and a sheath adjacent to the endotracheal tube; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve. The sheath includes a proximal and a distal end. The distal end of the endotracheal tube extends beyond the distal end of the sheath.

This aspect of the invention can have a variety of embodiments. In one embodiment, the step of stimulating the recurrent laryngeal nerve candidate includes applying electrical current to the recurrent laryngeal nerve candidate. The method can include inserting an additional endotracheal tube through the sheath to ventilate the subject. The airway management device can include one or more cuffs and the method can include actuating at least one of the one or more additional cuffs.

Another aspect of the invention provides a method of protecting a subject's recurrent laryngeal nerve during surgery, wherein the subject was previously intubated with an endotracheal tube using the airway management devices of the invention as herein described in all its various aspects and embodiments. The method includes: introducing an airway management device into the subject's larynx, the airway management device including an endotracheal tube having a proximal end and a distal end and a sheath adjacent to the endotracheal tube; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve, thereby protecting the subject's recurrent laryngeal nerve during surgery. The sheath includes a proximal and a distal end. The distal end of the endotracheal tube extends beyond the distal end of the sheath.

This aspect can have a variety of embodiments. The airway management device can include one or more cuffs and the method can include actuating at least one of the one or more additional cuffs.

Another aspect of the invention provides an endoscopic conduit including: a sheath having a proximal end and a distal end and a clip coupled to the distal end of the sheath. The clip is configured to straddle an endotracheal tube.

This aspect of the invention can have a variety of embodiments. The sheath can be configured to receive an endoscope. The endoscopic conduit can include an endoscope. The endoscopic conduit can include one or more actuatable cuffs. The one or more actuatable cuffs can be configured to dilate a subject's laryngeal tissues. The one or more actuatable cuffs can be configured to elevate a subject's epiglottis. The endoscopic conduit can include one or more valves configured to control actuation of the one or more actuatable cuffs. The one or more valves can be individually actuatable.

Another aspect of the invention provides a surgical kit including an endoscopic conduit and instructions for use. The endoscopic conduit includes: a sheath having a proximal end and a distal end and a clip coupled to the distal end of the sheath. The clip is configured to straddle an endotracheal tube.

This aspect can have a variety of embodiments. The clip can be a V-clip. The surgical kit can include an endoscope configured for passage through the sheath.

Another aspect of the invention provides an airway management device including an endotracheal tube and an endoscopic conduit of the invention as herein described in all its various aspects and embodiments.

Another aspect of the invention provides a method of identifying a subject's recurrent laryngeal nerve during surgery, wherein the subject was previously intubated with an endotracheal tube using the airway management devices of the invention as herein described in all its various aspects and embodiments. The method includes: introducing an endoscopic conduit into the subject's larynx, the endoscopic conduit includes a sheath having a proximal end and a distal end and a clip coupled to the distal end of the sheath; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve. The clip is configured to straddle the endotracheal tube.

Another aspect of the invention provides a method of protecting a subject's recurrent laryngeal nerve during surgery, wherein the subject was previously intubated with an endotracheal tube using the airway management devices of the invention as herein described in all its various aspects and embodiments. The method includes: introducing an endoscopic conduit into the subject's larynx, the endoscopic conduit including a sheath having a proximal end and a distal end and a clip coupled to the distal end of the sheath; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve, thereby protecting the subject's recurrent laryngeal nerve during surgery. The clip is configured to straddle the endotracheal tube.

Another aspect of the invention provides an airway management device including: an endotracheal tube having a proximal end and a distal end and a sheath adjacent to the endotracheal tube. The endotracheal tube includes at least one portion with a non-circular cross-section. The sheath includes a proximal end and a distal end. The distal end of the endotracheal tube extends beyond the distal end of the sheath.

This aspect can have a variety of embodiments. The non-circular cross-section is a cross-sectional shape selected from the group consisting of substantially oval and substantially triangular. The airway management device can include one or more cuffs coupled to the sheath.

Another aspect of invention provides a surgical kit includes an airway management device and instructions for use. The airway management device includes: an endotracheal tube having a proximal end and a distal end and a sheath adjacent to the endotracheal tube. The sheath includes a proximal and a distal end. The endotracheal tube includes at least one portion with a non-circular cross-section. The distal end of the endotracheal tube extends beyond the distal end of the sheath.

This aspect of the invention can include a variety of embodiments. The surgical kit can include an endoscope configured for passage through the sheath. The airway management can include one or more cuffs coupled to the sheath.

Another aspect of the invention provides a method of identifying a subject's recurrent laryngeal nerve during surgery using the airway management devices of the invention as herein described in all its various aspects and embodiments. The method includes: introducing an airway management device into the subject's larynx, the airway management device including an endotracheal tube having a proximal end and a distal end and a sheath adjacent to the endotracheal tube; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve. The sheath includes a proximal and a distal end. The endotracheal tube includes at least one portion with a non-circular cross-section. The distal end of the endotracheal tube extends beyond the distal end of the sheath;

This aspect of the invention can have a variety of embodiments. The airway management device can include one or more cuffs coupled to the sheath.

Another aspect of the invention provides a method of protecting a subject's recurrent laryngeal nerve during surgery using the airway management devices of the invention as herein described in all its various aspects and embodiments. The method includes: introducing an airway management device into the subject's larynx, the airway management device including an endotracheal tube having a proximal end and a distal end and a sheath adjacent to the endotracheal tube; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve, thereby protecting the subject's recurrent laryngeal nerve during surgery. The sheath includes a proximal and a distal end. The endotracheal tube includes at least one portion with a non-circular cross-section. The distal end of the endotracheal tube extends beyond the distal end of the sheath;

This aspect of the invention can have a variety of embodiments. The airway management device can include one or more cuffs coupled to the sheath.

Another aspect of invention provides an airway management device including: an endotracheal tube having a proximal end and a distal end and a sheath adjacent to the endotracheal tube. The sheath includes a proximal end and a distal end. The sheath includes a camera at the distal end. The distal end of the endotracheal tube extends beyond the distal end of the sheath.

This aspect of the invention can have a variety of embodiments. The sheath can include a light. The airway management device can include one or more cuffs coupled to the sheath.

Another aspect of the invention provides a surgical kit including an airway management device and instructions for use. The airway management device includes an endotracheal tube having a proximal end and a distal end and a sheath adjacent to the endotracheal tube. The sheath includes a proximal and a distal end. The sheath includes a camera at the distal end. The distal end of the endotracheal tube extends beyond the distal end of the sheath.

This aspect of the invention can have a variety of embodiments. The surgical kit can include an endoscope configured for passage through the sheath. The airway management device can include one or more cuffs coupled to the sheath.

Another aspect of the invention provides a method of identifying a subject's recurrent laryngeal nerve during surgery using the airway management devices of the invention as herein described in all its various aspects and embodiments. The method includes: introducing an airway management device into the subject's larynx, the airway management device including an endotracheal tube having a proximal end and a distal end and a sheath adjacent to the endotracheal tube, the sheath having a proximal and a distal end, wherein the sheath includes a camera at the distal end, and wherein the distal end of the endotracheal tube extends beyond the distal end of the sheath; visualizing the subject's vocal cords with the camera; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve.

This aspect of the invention can have a variety of embodiments. The airway management device can include one or more cuffs coupled to the sheath.

Another aspect of the invention provides a method of protecting a subject's recurrent laryngeal nerve during surgery using the airway management devices of the invention as herein described in all its various aspects and embodiments. The method includes: introducing an airway management device into the subject's larynx, the airway management device including an endotracheal tube having a proximal end and a distal end and a sheath adjacent to the endotracheal tube, the sheath having a proximal and a distal end, wherein the sheath includes a camera at the distal end, and wherein the distal end of the endotracheal tube extends beyond the distal end of the sheath; visualizing the subject's vocal cords with the camera; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve, thereby protecting the subject's recurrent laryngeal nerve during surgery.

This aspect of the invention can have a variety of embodiments. The airway management device can include one or more cuffs coupled to the sheath.

Another aspect of the invention provides an airway management device including: an endotracheal tube having a proximal end and a distal end, a sheath adjacent to the endotracheal tube, and a foam cuff coupled to the sheath.

This aspect of the invention can have a variety of embodiments. In one embodiment, the foam cuff expands upon contact with fluids.

Another aspect of the invention provides a surgical kit including an airway management device and instructions for use. The airway management device includes: an endotracheal tube having a proximal end and a distal end, a sheath adjacent to the endotracheal tube, and a foam cuff coupled to the sheath.

Another aspect of the invention provides a method of identifying a subject's recurrent laryngeal nerve during surgery using the airway management devices of the invention as herein described in all its various aspects and embodiments. The method includes: introducing an airway management device into the subject's larynx, the airway management device including an endotracheal tube having a proximal end and a distal end, a sheath adjacent to the endotracheal tube, and a foam cuff coupled to the sheath; visualizing the subject's vocal cords with the camera; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve.

Another aspect of the invention provides a method of protecting a subject's recurrent laryngeal nerve during surgery using the airway management devices of the invention as herein described in all its various aspects and embodiments. The method includes: introducing an airway management device into the subject's larynx, the airway management device including an endotracheal tube having a proximal end and a distal end, a sheath adjacent to the endotracheal tube, and a foam cuff coupled to the sheath; visualizing the subject's vocal cords with the camera; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve, thereby protecting the subject's recurrent laryngeal nerve during surgery.

Another aspect of the invention provides an endoscopic conduit including: a sheath having a proximal end and a distal end; a V-clip coupled to the distal end of the sheath; and one or more attachment devices for attachment to an endotracheal tube. The V-clip is configured to straddle an endotracheal tube.

This aspect of the invention can have a variety of embodiments. The endoscopic conduit can be pre-attached to the endotracheal tube.

Another aspect of the invention provides a surgical kit including an endoscopic conduit and instructions for use. The endoscopic conduit includes: a sheath having a proximal end and a distal end; a V-clip coupled to the distal end of the sheath; and one or more attachment devices for attachment to an endotracheal tube. The V-clip is configured to straddle an endotracheal tube

This aspect of the invention can have a variety of embodiments. In one embodiment, the surgical kit includes an endoscope configured for passage through the sheath.

Another aspect of the invention provides a method of identifying a subject's recurrent laryngeal nerve during surgery using the endoscopic conduits of the invention as herein described in all its various aspects and embodiments. The method includes: attaching an endoscopic conduit to an endotracheal tube, the endoscopic conduit including: a sheath having a proximal end and a distal end, a V-clip coupled to the distal end of the sheath, and one or more attachment devices for attachment to the endotracheal tube; inserting the assembled endoscopic conduit and endotracheal tube into the subject's larynx; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve. The V-clip is configured to straddle the endotracheal tube.

Another aspect of the invention provides a method of protecting a subject's recurrent laryngeal nerve during surgery using the endoscopic conduits of the invention as herein described in all its various aspects and embodiments. The method includes: attaching an endoscopic conduit to an endotracheal tube, the endoscopic conduit including: a sheath having a proximal end and a distal end, a V-clip coupled to the distal end of the sheath, and one or more attachment devices for attachment to the endotracheal tube; inserting the assembled endoscopic conduit and endotracheal tube into the subject's larynx; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve, thereby protecting the subject's recurrent laryngeal nerve during surgery. The V-clip is configured to straddle the endotracheal tube.

Another aspect of the invention provides an airway management device including: an endotracheal tube, a sheath adjacent to the endotracheal tube, and one or more EMG electrodes.

This aspect of the invention can have a variety of embodiments. The one or more EMG electrodes can be coupled to the endotracheal tube. The one or more EMG electrodes can be coupled to the sheath.

In one embodiment, the endotracheal tube includes a proximal end and a distal end, the sheath includes a proximal end and a distal end, and the distal end of the endotracheal tube extends beyond the distal end of the sheath.

Another aspect of the invention provides a surgical kit including: an airway management device and instructions for use. The airway management device includes: an endotracheal tube, a sheath adjacent to the endotracheal tube, and one or more EMG electrodes.

Another aspect of the invention provides a method of identifying a subject's recurrent laryngeal nerve using the airway management devices of the invention as herein described in all its various aspects and embodiments. The method includes: introducing an airway management device into the subject's larynx, the airway management device including an endotracheal tube, a sheath adjacent to the endotracheal tube, and one or more EMG electrodes; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; detecting an electrical signal with the one or more EMG electrodes; and identifying the recurrent laryngeal nerve candidate as the subject's recurrent laryngeal nerve.

Another aspect of the invention provides a method of protecting a subject's recurrent laryngeal nerve during surgery using the airway management devices of the invention as herein described in all its various aspects and embodiments. The method includes: introducing an airway management device into the subject's larynx, the airway management device including an endotracheal tube, a sheath adjacent to the endotracheal tube, and one or more EMG electrodes; introducing an endoscope into the sheath; visualizing the subject's vocal cords with the endoscope; identifying a recurrent laryngeal nerve candidate; stimulating the recurrent laryngeal nerve candidate; observing a movement in the subject's vocal cords with the endoscope; detecting an electrical signal with the one or more EMG electrodes; and identifying the recurrent laryngeal nerve candidate as the subject's recurrent laryngeal nerve, thereby protecting the subject's recurrent laryngeal nerve during surgery.

Another aspect of the invention provides an airway management device including: an endotracheal tube and one or more visualization devices coupled to the endotracheal tube.

This aspect of the invention can have a variety of embodiments. The one or more visualization devices can be cameras. The one or more visualization devices can be lenses. At least one of the visualization devices can be configured to provide a view of a subject's vocal cords during orotracheal intubation. At least one of the visualization devices can be configured to provide a view of a subject's vocal cords after orotracheal intubation.

Another aspect of the invention provides a surgical kit including: an airway management device and instructions for use. The airway management device includes an endotracheal tube and one or more visualization devices coupled to the endotracheal tube.

Another aspect of the invention provides a method of identifying a subject's recurrent laryngeal nerve using the airway management devices of the invention as herein described in all its various aspects and embodiments. The method includes: introducing an airway management device into the subject's larynx, the airway management device including an endotracheal tube and one or more visualization devices coupled to the endotracheal tube; visualizing the subject's vocal cords with the one or more visualization devices; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve.

Another aspect of the invention provides a method of protecting a subject's recurrent laryngeal nerve during surgery using the airway management devices of the invention as herein described in all its various aspects and embodiments. The method includes: introducing an airway management device into the subject's larynx, the airway management device including an endotracheal tube and one or more visualization devices coupled to the endotracheal tube; visualizing the subject's vocal cords with the one or more visualization devices; identifying a recurrent laryngeal nerve candidate; stimulating the recurrent laryngeal nerve candidate; observing a movement in the subject's vocal cords with the endoscope; and identifying the recurrent laryngeal nerve candidate as the subject's recurrent laryngeal nerve, thereby protecting the subject's recurrent laryngeal nerve during surgery.

Another aspect of the invention provides an endoscope including a visualization device and a V-clip coupled to the visualization device.

Another aspect of the invention provides a surgical kit including endoscope and instructions for use. The endoscope includes a visualization device and a V-clip coupled to the visualization device.

Another aspect of the invention provides a method of identifying a subject's recurrent laryngeal nerve during surgery, wherein the subject was previously intubated with an endotracheal tube using the endoscopes of the invention as herein described in all its various aspects and embodiments. The method includes: introducing an endoscope into the subject's larynx, the endoscope including a visualization device and a V-clip coupled to the visualization device introducing an endoscope into the sheath; visualizing the subject's vocal cords with the visualization device; identifying a recurrent laryngeal nerve candidate; stimulating the recurrent laryngeal nerve candidate; observing a movement in the subject's vocal cords with the visualization device; and identifying the recurrent laryngeal nerve candidate as the subject's recurrent laryngeal nerve.

Another aspect of the invention provides a method of protecting a subject's recurrent laryngeal nerve during surgery, wherein the subject was previously intubated with an endotracheal tube using the endoscopes of the invention as herein described in all its various aspects and embodiments. The method includes: introducing an endoscope into the subject's larynx, the endoscope including a visualization device and a V-clip coupled to the visualization device introducing an endoscope into the sheath; visualizing the subject's vocal cords with the visualization device; identifying a recurrent laryngeal nerve candidate; stimulating the recurrent laryngeal nerve candidate; observing a movement in the subject's vocal cords with the visualization device; and identifying the recurrent laryngeal nerve candidate as the subject's recurrent laryngeal nerve; thereby protecting the subject's recurrent laryngeal nerve during surgery.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1A depicts an embodiment of the invention including an endotracheal tube having a circular cross-section according to one embodiment of the invention.

FIGS. 1B, 1C, 1D, and 1E depict exemplary circular, oval, triangular, and hourglass endotracheal tube cross-sections, respectively.

FIGS. 1F, 1G, and 1H depict sheaths positioned internally to, externally to, and confluent with an endotracheal tube.

FIGS. 2, 3 and 4 depict an embodiment of the invention with an endoscope in place. This allows for passage of the endotracheal tube into the larynx, and visualization of the vocal cords with the endotracheal tube in place.

FIG. 5 depicts the use of an embodiment of the invention during neck surgery on a human subject.

FIG. 6A depicts an embodiment of the invention including a conduit.

FIG. 6B depicts the distal end of an embodiment of the invention in the larynx.

FIG. 6C is a cross-sectional depiction of the distal end of an embodiment of the invention.

FIG. 7 depicts a method of using an airway management device according to one embodiment of the invention.

FIG. 8 depicts a method of using an endoscopic conduit according to one embodiment of the invention.

FIG. 9 depicts an airway management device according to one embodiment of the invention.

FIGS. 10A and 10B depict an endoscope according to one embodiment of the invention.

### DETAILED DESCRIPTION

The instant invention is most clearly understood with reference to the following definitions:

As used in the specification and claims, the singular form “a,” “an,” and “the” include plural references unless the context clearly dictates otherwise.

A “health care provider” shall be understood to mean any person providing medical care to a patient. Such persons include, but are not limited to, medical doctors (e.g., surgeons, anesthesiologists, anesthetists, and the like), physician's assistants, nurse practitioners (e.g., an Advanced Registered Nurse Practitioner (ARNP)), nurses (e.g., nurse anesthetists), residents, interns, medical students, or the like. Although various licensure requirements may apply to one or more of the occupations listed above in various jurisdictions, the term health care provider is unencumbered for the purposes of this patent application.

A “subject” shall be understood to mean any organism capable of being intubated. Such organism include mammals such as humans, horses, cows, dogs, cats, rodents, and the like.

The following detailed description of certain embodiments of the invention is provided with reference to the accompanying drawings.

Embodiments of the invention are advantageously used during any neck surgery that involves dissection near the RLN and/or that puts the RLN at risk of injury during dissection and/or any surgery that involves the risk of difficult endotracheal intubation. Examples of such surgery include, but are not limited to, thyroidectomy, parathyroidectomy, branchial cleft surgery, neck dissection, cervical esophageal surgery, cervical spine surgery, carotid surgery, and sleep apnea surgery. A description of the device follows.

The invention provides various device embodiments and variations of these embodiments. Various embodiments of the device include one or more of:

a.) an endotracheal tube component,

b.) an endoscopy sheath component,

c.) an endoscope component, and/or

d.) an epiglottis cuff component

Each component will be described in detail, with reference to the accompanying figures.

### Endotracheal Tube Component

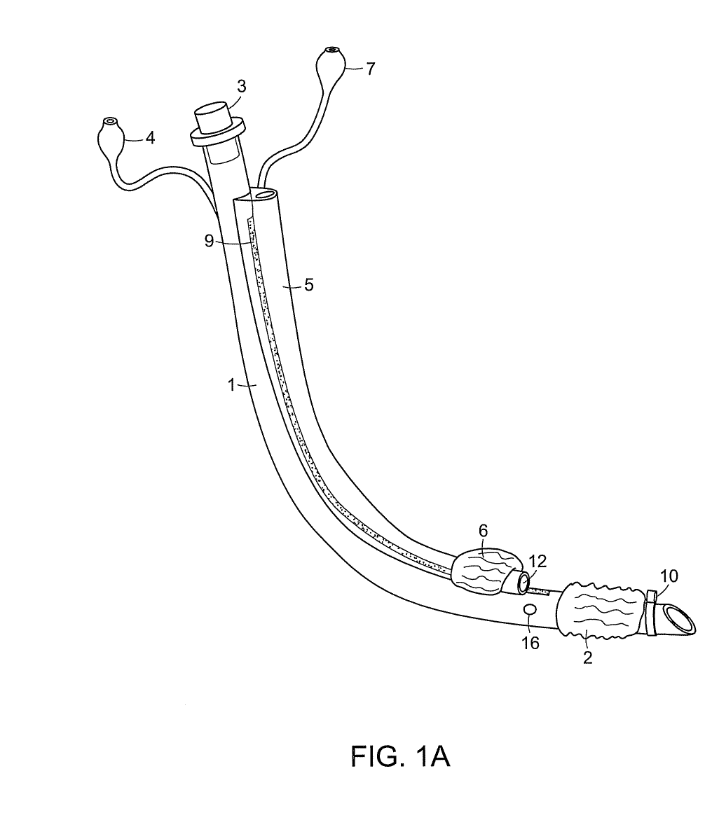

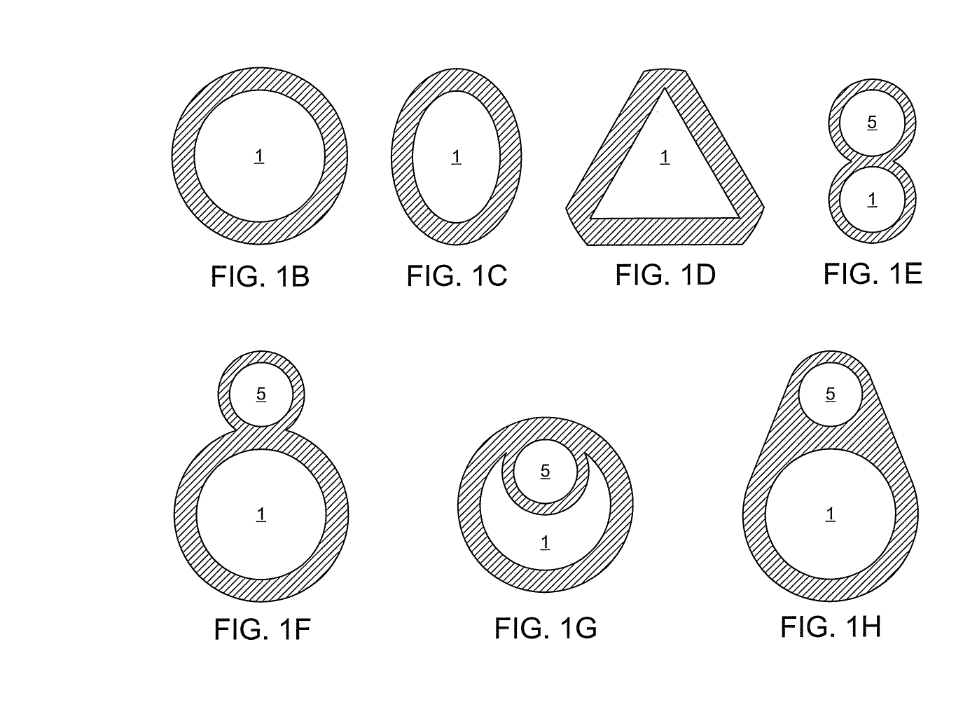

Referring now to FIG. 1, the endotracheal tube 1 can, in some embodiments, be constructed from standard medical grade plastic or rubber tubing. Endotracheal tube component 1 can include a flexible balloon cuff 2 with an adapter 3 to attach to a standard anesthetic breathing circuit. Additionally or alternatively, endotracheal tube component 1 can include a valve and tubing 4 to inflate the cuff 2.

The endotracheal tube 1 can be of variable length and diameters as is the case with standard endotracheal tubes. This variety of dimensions permits use of the device with a range of patient sizes from infants to large adults.

The endotracheal tube 1 can be flexible or stiff and can be armored for reinforcement. In armored tubing, one or more strands of a protective material are incorporated either internal, external, or within the tubing material to provide additional strength and kink resistance. Suitable materials include, but are not limited to, metals such as stainless steel or a nickel titanium alloy (also known as “nitinol”).

The endotracheal tube 1 can connected to the anesthetic tubing via a connector 3 and can be used for ventilation.

The endotracheal tube 1 can be of a cylindrical shape typically in use for current endotracheal tubes as depicted in FIGS. 1A and 1B. Alternatively, all or a portion of endotracheal tube 1 can have an oval, triangular or pinched (hourglass component) profile as depicted as depicted in FIGS. 1C, 1D, and 1E, respectively. As depicted in FIG. 1D, any sharp edges can be rounded over for easy and safe insertion and removal from the throat. A triangular shape would enhance rigidity in the spiral axis lessening the tendency of the endotracheal tube 1 to twist. Both the triangular and pinched versions would enhance positioning of the endotracheal tube 1 at the level of the vocal cords and facilitate proper orientation of the endotracheal tube 1 and endoscope.

### Endoscopy Sheath Component

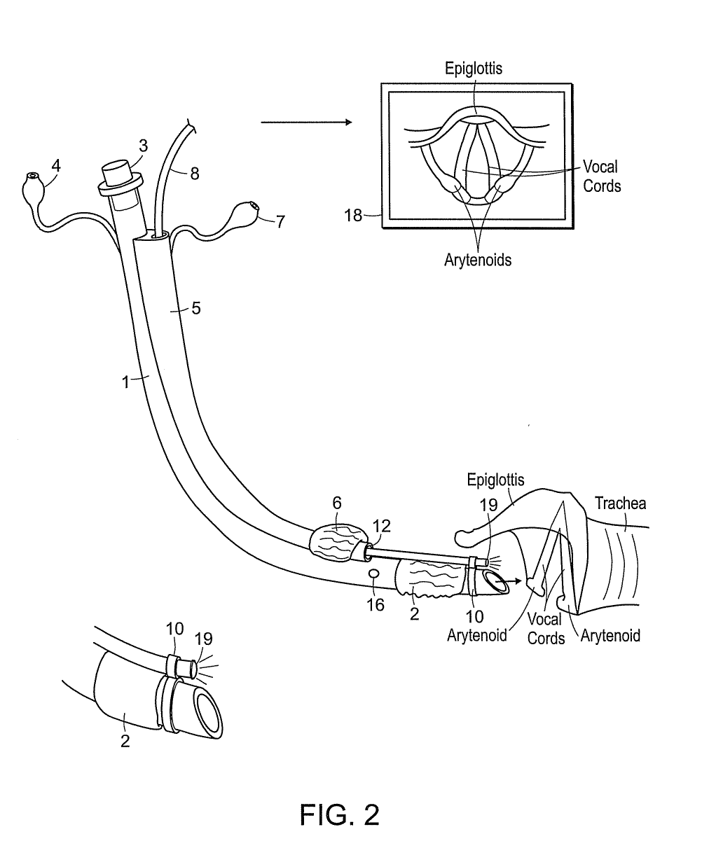

The endoscopy sheath 5 can be made of standard medical grade plastic. In some embodiments, endoscopy sheath 5 runs along with and approximately parallel to the endotracheal tube 1. As depicted in FIGS. 1F, 1G, and 1H, sheath 5 can be coupled internally to, externally to, or confluent with to endotracheal tube 1. The endoscopy sheath 5 functions to permit passage of an endoscope 8 (depicted in FIG. 2) in order to allow visualization of the arytenoids and/or vocal cords during surgery. In some embodiments, the endoscopy sheath 5 is positioned on the concave (superior) side of the endotracheal tube 1 as depicted in FIG. 1A. In other embodiments, the endoscopy sheath 5 can be positioned on the lateral or posterior side of one portion of the endotracheal tube 1 and on the concave side for another portion of the endotracheal tube 1. In some embodiments, endoscopy sheath 5 is fabricated from rigid or flexible plastic.

Endoscopy sheath 5 can be fabricated from or coated with a material that enables smooth passage of an endoscope 8 without excessive friction. Suitable low friction materials include polytetrafluoroethylene (PTFE)) and the like. Friction can be reduced by the application of a lubricant to the endoscope 8 and/or the sheath 5 before, during, or after insertion of the endoscope. Suitable lubricants include, but are not limited to, water soluble lubricants such as those sold under the SURGILUBE® trademark by Nycomed US Inc. of Melville, N.Y.

The caliber (internal diameter) of endoscopy sheath 5 may range from about 2 mm to about 10 mm to permit passage of the endoscope component 8 without binding. The length of the endoscopy sheath 5 can vary from about 1 cm to about 40 cm to permit a variety of length and diameter endoscopes 8 to be used. This range of lengths is deliberately wide to permit passage of a variety endoscopes 8 either briefly to allow a single view of the larynx, or permit passage of an endoscope 8 that is left indwelling during surgery to allow ongoing monitoring throughout the entire operation.

The sheath end point 12 is designed such that the endoscope 8 when passed beyond the end point 12 will permit a satisfactory view of the arytenoids and/or vocal cords. In some embodiments, the endpoint 12 ends about zero to about 10 cm proximal to the proximal end of the endotracheal tube cuff 2. For example, endpoint 12 can be between about 0 cm and about 1 cm, between about 1 cm and 2 cm, between about 2 cm and about 3 cm, between about 3 cm and about 4 cm, between about 4 cm and about 5 cm, between about 5 cm and about 6 cm, between about 6 cm and 7 cm, between about 7 cm and about 8 cm, between about 8 cm and about 9 cm, or between about 9 cm and about 10 cm from the proximal end of cuff 2. This range is deliberately wide so that the portion of endoscope 8 extending beyond the sheath end point 12 is sufficient to permit laryngeal visualization without obstruction by adjacent structures such as the epiglottis, and so that the endoscope 8 does not extend so far as to lead to an overly narrow field preventing view of glottic aperture or so far as to interfere with the airway.

In some embodiments, the sheath 5 includes a camera, lens, and/or light source combination at the end point 12. The lens can be fabricated from a variety of materials including “fog-free” materials known to those of ordinary skill in the art and can have a variety of focal lengths. This may include a video camera with a charge-coupled device (CCD) or complementary metal-oxide-semiconductor (CMOS). The light source can be a halogen light source, a fiber optic light source, an LED light source, and the like. This camera/lighting combination may be disposable or reusable. It may be integrated or removable. The camera and light in this embodiment are connected via a cable or wire to an external control module for video signal processing and as a light source for fiber optic transmission, or power source for LED transmission. In other embodiments in which the camera/light are attached at end point 12, the video signal can be transmitted from the camera to the video signal processor wirelessly, such as by radio frequency signals.

Referring to FIG. 1A, the sheath 5 may also include one or more shape or batten components 9 which serve to stiffen the endotracheal tube 1 especially but not limited to the spiral axis. The effect in particular is to prevent a twisting of the endoscope sheath 5. Shape or batten components 9 can be can a rigid material such as metal (e.g., a malleable metal such as aluminum) that can be shaped prior to insertion and maintain this shape after insertion. In this manner, the sheath endpoint 12 and indwelling endoscope 8 can remain in place without dislodgement after placement. For example, through the shape or batten components 9 of the sheath, once the endoscope is in place so that the vocal cords are visible, moving the endotracheal tube 1 or sheath 5 would not result in loss of view of the vocal cords. This shape may include but is not limited to, a cylindrical shape which is circular or oval in cross section, a rectangular or box shape, which is square or rectangular in cross section, or a larger rectangular shape which includes the endotracheal tube 1 within the endoscope sheath 5.

The endotracheal tube 1 can also include a fastener 10. The fastener 10 attaches to the tube 1 between the cuff and the distal tip of the tube and includes a loop shaped portion that can hold and immobilize the endoscope 8. The fastener 10 can be located about 0 to about 5 cm from the distal tip of the endotracheal tube 1. Fastener 10 can be made of plastic materials and can be hard, flexible, elastic, or soft. FIG. 2 depicts an endoscope 8 held in the fastener 10.

### Endoscope Component

Endoscope 8 enables visualization of the arytenoids, vocal cords, and other muscles of the larynx before, during and/or after surgery. In this manner the surgeon, anesthetist, and/or anesthesiologist can determine if laryngeal muscle motion and RLN function is intact. When the RLN is not being stimulated and the patient is not excessively anesthetized, the patient may have spontaneous respiration. This state permits visualization of the patient's normal laryngeal muscle motion, allowing determination of whether both RLNs and vocal cords are functioning normally before and after surgery. During surgery, use of the endoscope 8 permits visualization of the vocal cords and laryngeal muscles during stimulation of the RLN and other structures. This allows determination of whether a nerve candidate is in fact the RLN and whether the RLN is functioning.

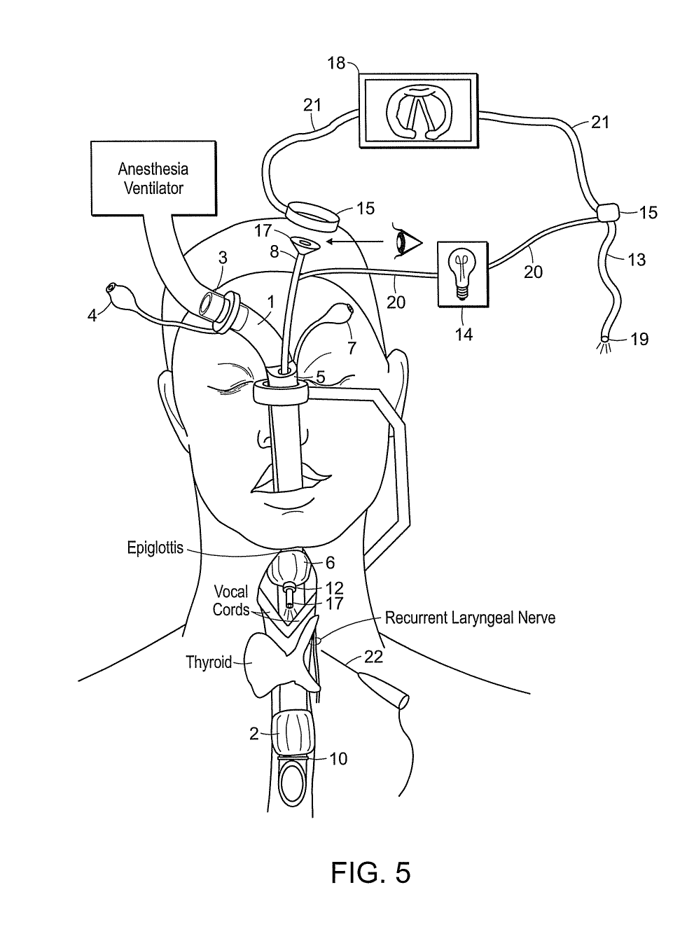

Embodiments of the invention may use a wide variety of commercially available endoscopes 8 or an endoscope 13 (described herein and depicted in FIG. 5).

One embodiment of the invention incorporates a rigid straight endoscope ranging in width from about 2 to about 10 mm and of a range of lengths from about 1 cm to about 30 cm, with lens angles ranging from zero to 45 degrees. This range permits the use of existing endoscopes as well as a small micro-endoscope that is indwelling in the endoscope sheath 5 and connected to a light source 14 and camera 15 by wires passing through the sheath 5.

Another embodiment of the invention incorporates a rigid or semi-rigid curved endoscope ranging in width from about 2 to about 10 mm and of varied lengths from about 1 to about 60 cm with lens angles ranging from zero to 45 degrees. The curve of the endoscope would conform to the curve naturally present between the patient's mouth and the entry point 12 when the device is in place and may permit easier passage than with a straight rigid endoscope.

Another embodiment of the invention incorporates a flexible endoscope ranging in width from about 2 to about 10 mm and of varied lengths. This endoscope may be of the “steerable” or non-steerable variety. This range permits the use of existing flexible endoscopes as well as a specialized endoscope wand. This endoscope wand would attach to a light source 14 and chip camera 15 as depicted in FIG. 5.

Another embodiment of the invention incorporates standard endoscopes and chip cameras that are currently available for visualizing the vocal cords and/or larynx.

The endoscope proximal end can include an ocular 17 to view the larynx directly or to which a camera 15 may be attached for viewing over a video monitor 18. Alternatively, the endoscope may attach to a cable 21 for attachment directly to surgical monitors 18. In such an embodiment, a camera (e.g. a chip camera) can be located within the endoscope 8, such as at the proximal or distal end or intermediate to the proximal and distal ends. The camera may output video signals in a proprietary or standard video format. The endoscope 8 can connect with monitor 18 using proprietary or standard connections such as RF connectors, component video (e.g. NTSC, PAL, or SECAM), S-Video, component video, Serial Digital Interface, FireWire, High-Definition Multimedia Interface (HDMI), USB, or Ethernet. Alternatively, monitor 18 or a video processing unit can be connected to the endoscope 8 by one or more fiber optic strands, which relay an image from the distal end of the endoscope 8.

As described herein, in another embodiment, the endoscope camera, lens and light supply can be incorporated into the endotracheal tube 1 at end point 12 rather than passed via endoscopy sheath 5.

Surgical monitor 18 can have many forms. In some embodiments, surgical monitor 18 is a television or a computer monitor (e.g., a cathode-ray tube (CRT), liquid crystal display (LCD), plasma screen, and the like). In other embodiments, the monitor 18 is a general purpose computer such as a desktop, a laptop, a tablet, and the like. The surgical monitor 18 can be configured or coupled to a device for capturing and/or recording video and/or images transmitted from the endoscope 8.

Surgical monitor 18 can be located in the operating room or in another location. In some embodiments, the surgical monitor 18 is wall-mounted. Additionally or alternatively, the surgical monitor 18 can be a relatively small LCD screen coupled with the endoscope 8 or endotracheal tube 1 or other components of the invention. Such an embodiment is advantageous as it allows the surgeon to visualize the vocal cords during the insertion of an endotracheal tube. In other embodiments, the surgical monitor is mounted on a cart so that surgeon, anesthetist, or anesthesiologist can easily view the larynx during surgery.

The invention also provides an endoscope 8 for use with the devices herein. The endoscope 8 can include components commonly found in medical endoscopes such a lens 19, a fiber optic light cable 20 for carrying light from light source 14, a fiber optic image cable 21 for carrying the optical image to surgical monitor 18. Like some medical endoscopes, one variation of the endoscope 8 includes a channel for suction or instillation of liquids and/or a component for manipulating the distal tip of the endoscope 8. Alternatively, the endoscope sheath 5 can include one or more channels for suction or installation of fluids and/or for manipulating the end of endoscope 8 and/or camera lens 19.

The endoscope 8, chip camera 15, light source 14, and interfaces can include a number of variations that would permit either a set of cables connecting the endoscope 8 to the monitor 18 and light source 14, or a lightweight, battery-operated light source and radio frequency camera attached directly to the end of the endoscope 8 similar to those currently available. The length of the endoscope 8 can range from about one cm to about 60 cm. This broad range permits either a very small endoscope 8 including the light source 14 and chip camera 15 entirely within in the endoscope sheath 5 or a longer endoscope 8 with the lens 19 and fiber optic cables 20, 21 in the endoscope sheath 5 and the light source 14 and chip camera 15 external to the sheath 5.

The light source 14 can be a standard halogen-type light source as commonly used for endoscopes, or a smaller battery-powered light source recently developed for portable endoscopes such as one or more light-emitting diodes (LEDs). The chip camera 15 can be of a type commonly used in medical endoscopes. The interface between the light source 14 and the fiber optic cable 20 can be of the standard type of cable used in endoscopes or a connector as commonly used between small battery powered lights and fiber optic light cables. The interface between the chip camera 15 and the monitor 18 can be a standard type of cable used in endoscopes or may be of a radiofrequency transmitting variety currently available commercially. Any of the endoscopes 8 may have any of a variety of systems, clamps, and adhesive straps to hold it in place during the monitoring process or can be inserted for a brief view and removed immediately after viewing.

### Epiglottis Cuff Component

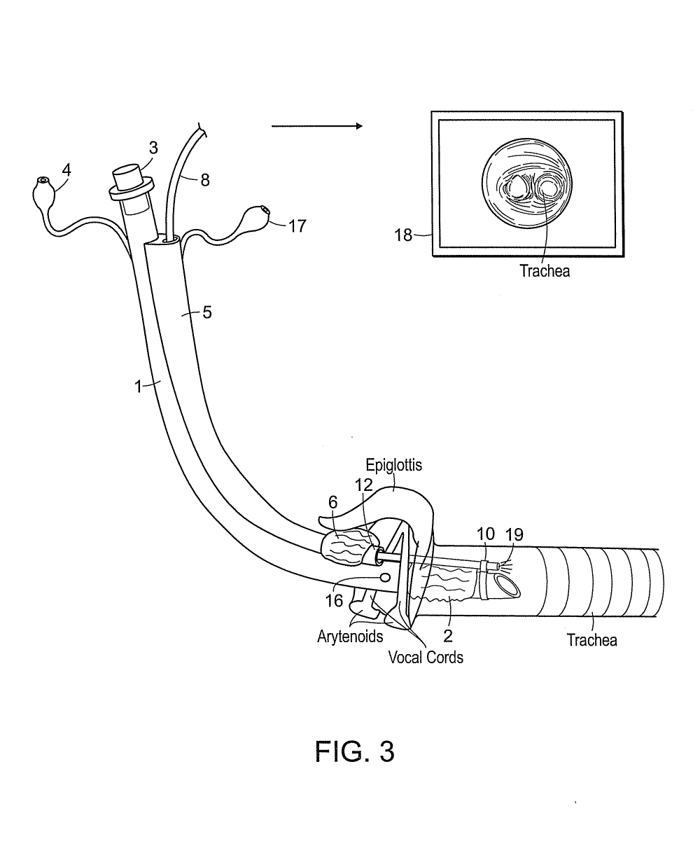

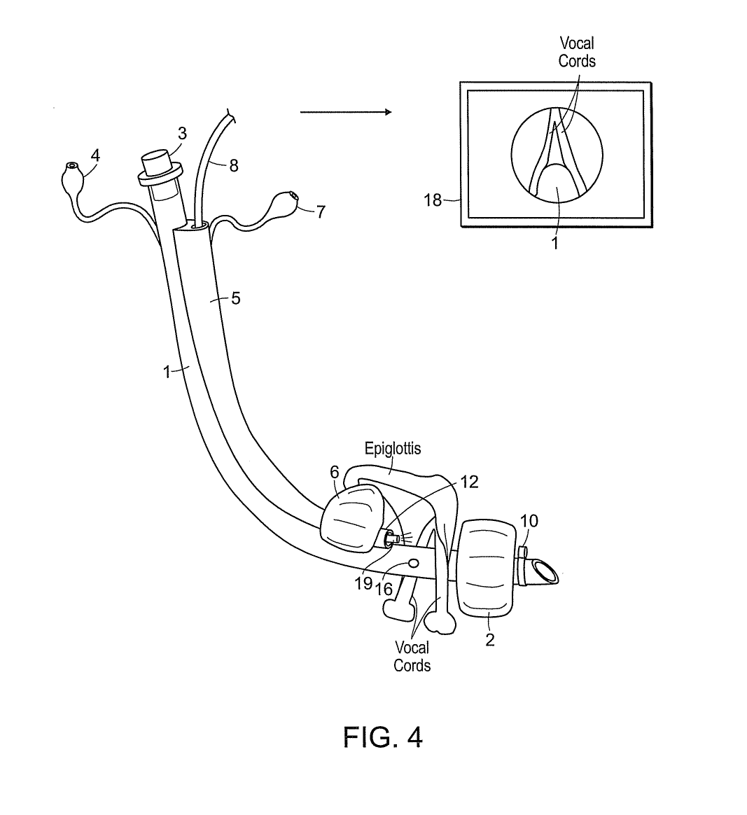

Referring to FIGS. 1A and 2-5, a cuff 6 can be made of a medical-grade plastic, sponge or foam material and can be of variable length, widths, thicknesses and curvatures, to permit satisfactory fits of adult and pediatric patients of varying sizes. The cuff 6 can be positioned on the outside of the sheath 5 and/or endotracheal tube 1 such that its actuation will elevate the epiglottis, permitting optimal visualization of the vocal cords with the endoscope 8.

Referring again to FIGS. 1A and 2-5, the cuff 6 can be actuated via a tubing and valve 7 as is typically used for inflation of medical balloon cuffs. As will be appreciated by one of skill in the art, the tubing used to actuate cuff 6 can be positioned internally, externally, and/or incorporated within endotracheal tube 1. In embodiments in which the cuff 6 is made of foam or sponge, the foam or sponge can be a variety that inflates upon contact with fluids. Suitable materials for a self-inflating cuff 6 include polyvinyl acetate foam as discussed in Arie Gordon et al., “Effect of a Novel Anatomically Shaped Endotracheal Tube on Intubation-Related Injury,” 136(1) Arch. Otolaryngol. Head Neck Surg. 54-59 (January 2010). In still other embodiments, cuff 6 can be a rigid or semi-rigid material cuff that is not actuated, but rather maintains a substantially similar shape during insertion.

The cuff 6 can be located in the midline or to either side of the sheath 5 and at a range of distances of 0 to 6 cm from the endpoint of the sheath 12 to permit elevation of a wide variety of shapes and sizes of epiglottis structures as may be present in children and adults. In one embodiment, the cuff 6 can extend distally beyond the endpoint of the sheath 5 a distance of zero to 6 cm, ending just proximal to the endotracheal tube cuff 2 and overhang the sheath opening 12 to ensure adequate elevation of short or thick epiglottis structures.

Cuff 6 can also splay and dilate the larynx and hypopharynx in a manner that prevents the soft tissues of the throat from collapsing and obstructing visualization of the vocal cords with the endoscope 8.

Cuff 6 also enables repositioning of the endoscope 8 and/or lens 19 relative to the vocal cords through inflation of a cuff 6 or multiple cuffs 6 on the various sides of the sheath 5. In embodiments with multiple cuffs 6, one or more valved inflation points 7 can be provided to allow for individual adjustment of one or more cuffs 6. Such individual adjustment allows for both the lateral movement of the endoscope 8 relative to the larynx and hypopharynx tissues by increasing pressure/volume of cuffs 6 on a first side of the sheath 5 and/or decreasing pressure/volume of cuffs 6 on a second side of the sheath 5. Furthermore, one or more cuffs 6 can be adjusted to promote the desired stenting and/or dilation of the larynx.

### Endoscopic Conduit

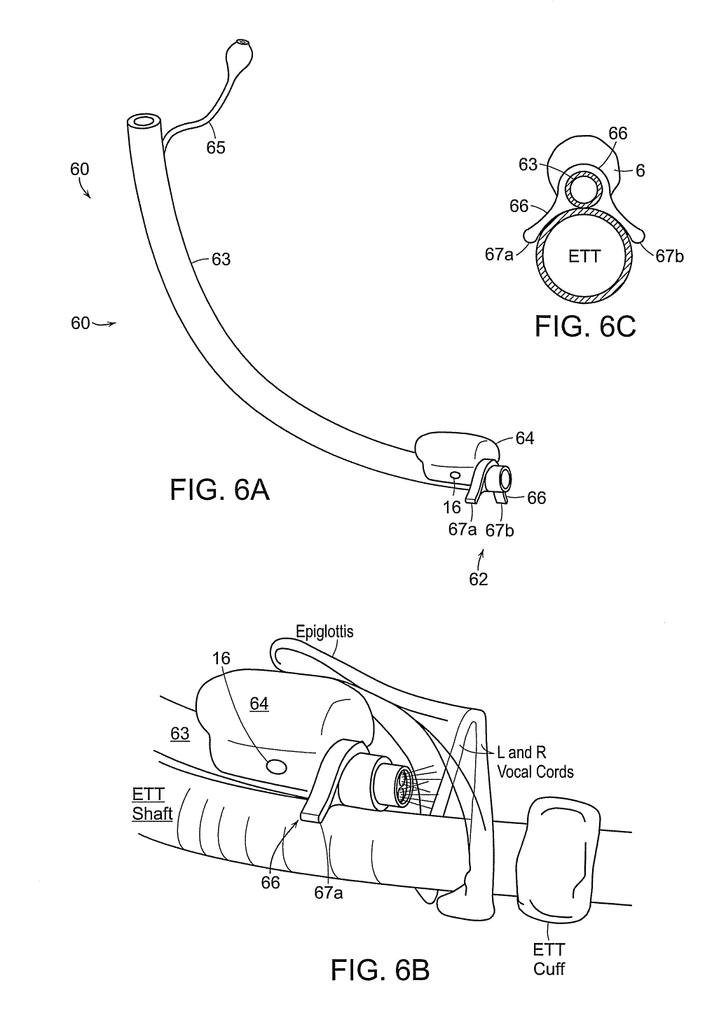

Referring now to FIG. 6, an endoscopic conduit 60 having a proximal end 61, a distal end 62, and a sheath 63 can be used to visualize the vocal cords for motion in patients who are already intubated with an endotracheal tube. Conduit 60 can perform the same or similar functions as sheath 5 discussed herein. For example, sheath 63 can receive an endoscope 8. The sheath 63 can include one or more channels for the application of solutions such as saline or defogging solution and to evacuate fluid and/or secretions.

In one embodiment, the sheath 63 can include attachment devices such as adhesive tapes, elastic bands, or hook-and-loop straps that permit the sheath to attach to an endotracheal tube. The distal end 62 of the endoscopic conduit 60 can include a cuff 64 designed to dilate the laryngeal tissues and elevate the epiglottis to optimize the view of the vocal cords seen with the endoscope 8 in the sheath 63. The cuff 64 can be inflated using a standard valve 65.

In some embodiments, an endoscopic conduit includes a clip 66 such as the “V-clip” 66 depicted in FIGS. 6A-6C. The clip 66 can be made from metals, plastics, hard plastics, rubbers, and the like. Clip 66 can be attached to the distal end 62 of the sheath 63 like a ring on a finger or through other techniques such as the use of fasteners (e.g., nails, screws, rivets, bolts, and the like), adhesives, welding, and the like. The clip 66 can include two “wings” 67 like the wings on the letter “V.” Although the figures depict a V-clip (i.e., V-shaped), the clip can take any suitable shape as long as the shape is configured to straddle an endotraecheal tube.

When the endoscopic conduit 60 is inserted into the intubated patient's mouth, the wings of the clip 66 straddle the endotracheal tube. This allows the endoscopic conduit 60 to ride atop the endotracheal tube as it is advanced, keeping the endoscopic conduit 60 centered atop the endotracheal tube and directing it toward the vocal cords. The clip 66 need not attach firmly to the endotracheal tube, although in certain designs it can. For example, clip 66 can substantially surround the endotracheal tube.

In order to reduce friction when the clip 66 is advanced down an endotracheal tube, clip 66 can, in some embodiments, be fabricated from and/or coated with a non-stick material such as PTFE.

### Incorporation of EMG Electrodes

Additionally or alternatively and as depicted in FIGS. 1A, 2, 3, 4, 6A, and 6B, endotracheal tube 1, sheath 5, endoscope 8, and/or surgical conduit 60 can include one or more electromyography (EMG) electrodes 16 configured to monitor the electrical signals triggered by stimulation and/or firing of the RLN. Such electrical signals can be displayed on a surgical monitor 18, which can be modified to include an oscilloscope and/or an oscilloscope display.

Endotracheal Tubes Incorporating Visualization Devices and/or Electrodes

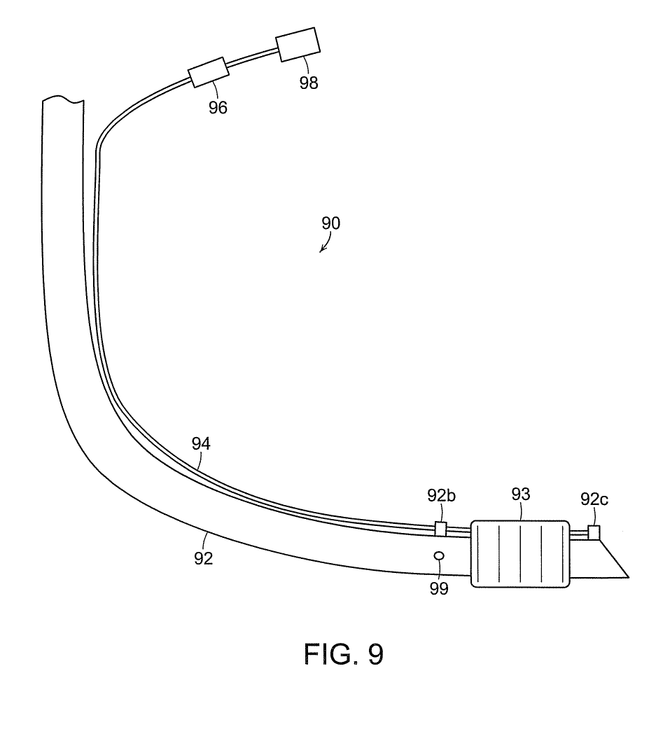

Referring now to FIG. 9, another embodiment of the invention provides an airway management device 90 including an endotracheal tube 92 and one or more integrated visualization devices 92a, 92b as described herein.

Visualization devices 92a, 92b can be accessed by a monitor 98 via a communication conduit 94 and one or more connectors 96 as discussed herein. In embodiments in which the visualization devices 92a, 92b are cameras, communication conduit 94 can include one or more wires for transmitting power and signals. In embodiments in which the visualization devices 92a, 92b are lenses, communication conduit can include one or more glass rods and/or glass fibers for the transmission of light and images.

Airway management device can include cuff 93, EMG electrode 99, and/or other features discussed herein in the context of other embodiments of the invention.

### Endoscope Incorporating V-Clip

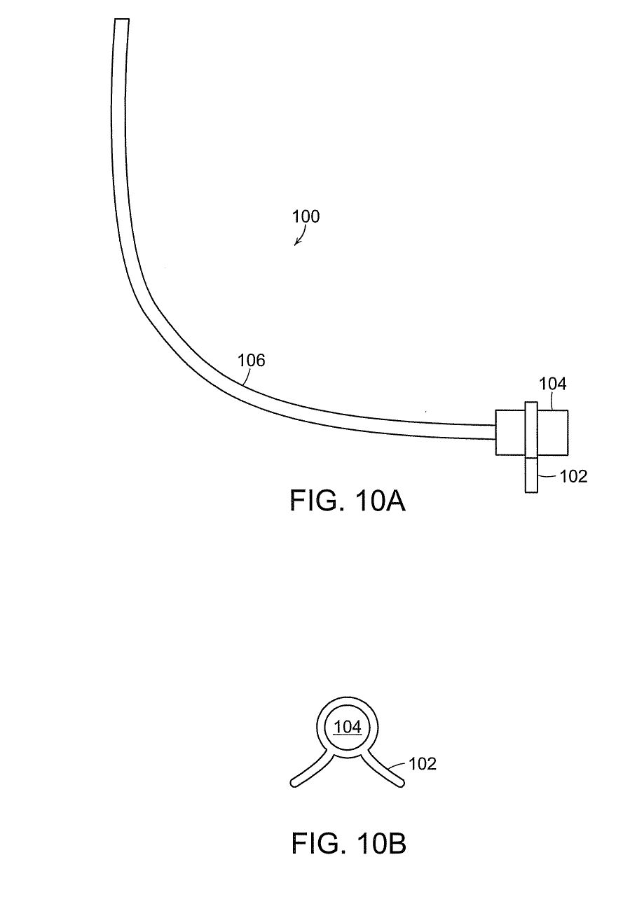

Referring now to FIG. 10, another embodiment of the invention provides an endoscope 100 including a V-clip 102 as described herein. Such an embodiment allows for the endoscope 100 to be inserted along an endotracheal tube without the need for the endoscopic conduits described herein.

As will be appreciated by those of ordinary skill in the art, endoscope 100 can include a visualization device 104 and a communications conduit 106 as discussed herein.

### Methods of Use of Various Embodiments of the Invention

It is very important that the surgeon, anesthetist, and/or anesthesiologist know prior to neck surgery whether the patient's arytenoids and/or vocal cords function normally. Currently, the arytenoids and vocal cords are assessed by some surgeons in the office with a mirror exam (indirect laryngoscopy), or with fiber optic endoscopy, which can be imprecise or uncomfortable. Many surgeons, including most general surgeons, are not adept in these techniques of laryngeal assessment and do not assess vocal function adequately prior to incision. This can be a problem in a patient who may have a pre-existing vocal cord paralysis, fixation or airway compromise that is not detected prior to surgery.

First, patients who have a single paralyzed nerve rely upon the opposite functioning side for respiration. Injury during surgery or other loss of function of this side jeopardizes the airway. Wakeup from anesthesia in such patients can be hazardous. In addition, if a pre-existing vocal paralysis is not identified prior to surgery, when it is discovered it may be incorrectly deemed a complication of the recent neck surgery.

Furthermore, vocal cord paralysis in the setting of a thyroid cancer indicates a worsened prognosis and may require a change in the surgical plan. For all of these reasons, the surgeon, anesthetist, and/or anesthesiologist need to know the level of function of the vocal cords and RLN prior to surgery. The devices provided herein allows the surgeon and anesthesiologist to assess vocal cord motion prior to neck incision, ensuring that it is normal, or alternatively to be aware that it is abnormal and make appropriate adjustments.

Likewise, it is imperative that the surgeon, anesthetist, and/or anesthesiologist know at the conclusion of neck surgery whether the patient's vocal cords move normally and that the RLN is functioning. Currently, this is assessed by some surgeons in the office with a mirror exam or fiber optic endoscopy which can be imprecise or uncomfortable. Many surgeons, including most general surgeons, are not adept in these techniques of laryngeal assessment and do not assess vocal cord mobility postoperatively. The devices provided herein allows the surgeon and anesthesiologist to assess vocal cord motion at the conclusion of surgery and prior to wakeup from general anesthesia, ensuring that it is normal, or alternatively to be aware that it is abnormal and make appropriate adjustments to manage a vocal cord(s) that is not functioning adequately. This also permits documentation that vocal cord and RLN function were preserved during surgery, and that any subsequent change in vocal mobility is not attributable to surgery.

Also, it is essential for surgeons to know during neck surgery the location of the RLN. Identification of the RLN allows the surgeon to avoid or minimize manipulation of and injury to the RLN and can increase the chance that it will be preserved and its function maintained during surgery. Identification of the RLN also permits more complete removal of the adjacent thyroid tissue with less fear or risk of accidental RLN injury.

Some surgeons identify the RLN visually only, i.e. the surgeon assumes that tissue that appears to be the RLN is the RLN. However, this method does not ensure adequate RLN identification because other structures such as blood vessels may have a similar appearance or the surgeon may be identifying only a branch of the RLN, believing it to be the entire RLN. Other surgeons identify the RLN by stimulating the structure with electric current, then monitoring the laryngeal muscles and vocal cord for a response to this stimulation. Suitable electrical stimulation devices are sold under the NEUROSIGN® trademark by The Magstim Company Limited of Carmarthenshire, Wales, United Kingdom. See Michael Hermann, et al., “Neuromonitoring in Thyroid Surgery: Prospective Evaluation of Intraoperative Electrophysiological Responses for the Prediction of Recurrent Laryngeal Nerve Injury,” 240(1) Ann. Surg. 9-17 (2004). Methods for monitoring the laryngeal muscles include: palpation of the laryngeal muscles for motion, monitoring the muscles for electrical or muscle potentials with electrodes, visually monitoring the muscles for motion, and the like.

Embodiments of the invention allow monitoring of the muscles visually for motion when the RLN is stimulated. Such embodiments are advantageous over other systems for at least the following reasons.

First, monitoring of the laryngeal muscles through palpation is imprecise and requires experience to be accurate. Only one person can palpate for a laryngeal muscle twitch at a time, which prevents verification by others. Proper placement of the palpating finger requires additional dissection to expose the posterior laryngeal muscles. This entails increased surgical time, risk of bleeding and injury to the RLN. If the palpating finger is not in the proper, precise location, the muscle twitch can fail to be palpated (false negative) and the RLN can be inadvertently injured. Placement of the palpating hand and finger often obscures the view of the RLN in the surgical field making accurate RLN stimulation more difficult. If the RLN is weak but intact, the degree of muscle motion triggered by stimulation may not be sufficient to create a palpable twitch, making the technique inaccurate. Also, the palpation technique cannot be employed during minimally invasive or endoscopic approaches to neck surgery, because these approaches do not allow surgical exposure sufficient to palpate the posterior laryngeal muscles.

Embodiments of the invention are advantageous because they require no additional dissection nor alteration of the surgical field from its state during dissection. Multiple persons can view the larynx for motion during RLN stimulation providing independent verification. Identifying muscle and vocal cord motion does not require experience and can be delegated to any medical personnel, permitting non-surgeons or less-experienced surgeons to judge the response to nerve stimulation while the surgeon stimulates the nerve. This is not possible with the palpation technique because both nerve stimulation and muscle palpation require experience, and in most cases, two experienced surgeons. If the RLN is weak but intact, diminished vocal cord motion may be visible yet not palpable, therefore, again the risk of inadvertent nerve injury through false negative response is reduced.

Second, monitoring of the laryngeal muscles through electrical or muscle potentials requires placement of electrodes internal or external to the larynx. If the electrodes are not properly placed, or if they shift during surgery, they will provide a false negative result, potentially leading to inadvertent nerve injury. In most techniques currently used, the surgeon will be unaware of a shift of the electrodes, greatly increasing the risk of a false negative response. Proper placement of the electrodes requires additional skill, as does interpretation of the electrical tracings of the electrodes. Improper placement will lead to a false negative result and increased risk of nerve injury. Motion of the larynx and neck during surgery can stimulate the electrodes even through the RLN is not being directly stimulated causing false positive responses. False positive responses cause undue concern over the status of the RLN, thereby slowing surgical speed.

Embodiments of the invention do not require placement of special devices or electrodes. Their design allows easy passage of an endoscope by untrained personnel. While visualization and determination of vocal cord motion requires some medical knowledge, the person doing this interpretation can be the surgeon who views the larynx on a monitor while the endoscope is passed by a nurse, technician, anesthetist, or resident. If the endoscope is not in proper place, the poor view will be immediately apparent unlike shifted or unattached electrodes, thereby reducing the false negative rate. Unlike monitoring systems based solely on electrodes, a shift in the position of the devices provided herein will be immediately apparent to the surgeon because such movement will result in a loss of view of the vocal cords, thus reducing the likelihood of false negatives. False positive rates are not an issue as the vocal cord will not twitch with motion of the neck or larynx.

Third, monitoring of the laryngeal muscles visually with endoscopes has generally required disconnection of the laryngeal mask from the ventilator and passage of the endoscope via this opening into the larynx. This causes wide leakage of the associated anesthetic gases, and simultaneous ventilation of the patient is not possible. As a result, nerve monitoring can only be done for brief periods, because it requires interruption of ventilation. A flexible endoscope can be passed through a side port on the laryngeal mask airway, but existing laryngeal mask airway designs often prevent easy passage of the endoscope or do not permit viewing of the vocal cords because of the location of the opening within the mask airway. The tubing may be too narrow, the bend in the tubing too severe, or plastic components intrude and prevent smooth endoscope passage. Current systems require skill on the part of the personnel passing the endoscope, otherwise the larynx will not be easily and properly viewed. Alternatively, individuals have passed endoscopes in patients intubated with standard endotracheal tubes. In such techniques, accurate passage of the endoscope is difficult because of the collapse of the laryngeal tissues.

Embodiments of the invention including both a visual-based and electrode-based vocal cord monitoring provide complimentary methods of RLN monitoring. Such embodiments permit the surgeon to assess RLN function by visible vocal cord motion, muscle twitches, or both. As described, the visualization component also ensures that the electrode component remains in the proper portion of the larynx, decreasing the risk of false negatives results during RLN stimulation.

Embodiments of the invention are designed to permit precise passage of the endoscope to a point within the airway permitting excellent vocal cord visualization, even when performed by minimally-trained personnel. This is important because the surgeon and anesthesiologist may be otherwise occupied in stimulating the RLN and monitoring the vocal cord for motion. By using embodiments of the invention, the surgeon and anesthesiologist need not be involved in passage of the endoscope as well. Embodiments of the invention also permit ongoing monitoring of the larynx during surgery—something that is not possible with existing laryngeal mask airways.

Some surgeons visualize the larynx during neck surgery by placing a laryngeal mask airway above an endotracheal tube, then passing the endoscope down the laryngeal mask airway. In such cases, the laryngeal mask functions as a conduit for the endoscope and is not used for ventilation. Arytenoid motion is monitored with the endotracheal tube in place. This design however, is cumbersome to place as currently available endoscopes are not well suited for passage through laryngeal masks for long periods. Furthermore there are no well designed devices to immobilize the endoscope for the long periods required for surgery. As a result, the devices have to be passed and removed repeatedly during the operation. The laryngeal mask in these cases must be passed either to the right or the left of the endotracheal tube, so the arytenoids and vocal cord on one side are seen less well than those on the other side.

Embodiments of the invention (e.g., the embodiment depicted in FIG. 6) permit an indwelling option for laryngeal monitoring in the presence of an endotracheal tube. Because the endoscope depicted in FIG. 6 is passed alongside and atop the endotracheal tube 1, the endoscopic conduit 60 lies midline and both vocal cords are visualized equally well. The suction channel also permits evacuation of saliva that otherwise pools and obscures the view during surgery.

Patients undergoing neck surgery receive general anesthesia through an endotracheal tube to connect the patient's respiratory tract with the ventilator. Because embodiments of the invention also permit visualization of the vocal cords, the anesthesiologist is able to pass the endotracheal tube 1 between the vocal cords under direct visualization of the endoscope 8. This allows the anesthesiologist to pass an endotracheal tube 1 and adjust it using embodiments of the invention at any point during the operation without losing the ability to monitor vocal cords. Additionally, the position of the lens 19 can be adjusted without altering the position of the endotracheal tube 1.

Embodiments of the invention can be inserted in a variety of methods.

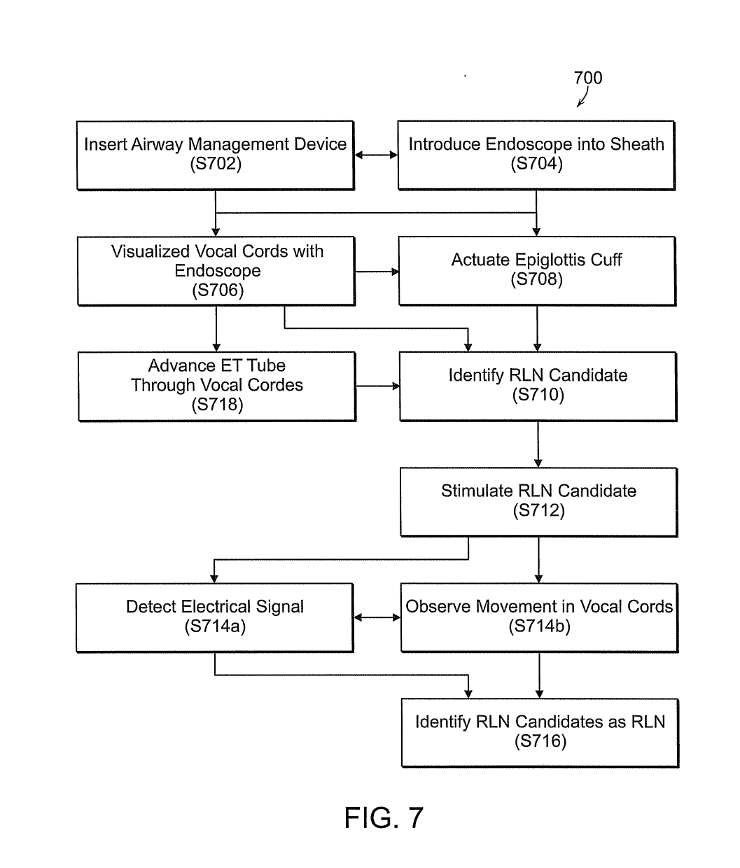

As depicted in FIG. 7, in one method 700, a health care provider introduces the device by passing the device as one would pass any endotracheal tube using a laryngoscope in the standard fashion (S702). During insertion of the tube 1, the endoscope sheath 5 is kept in the anterior midline and advanced such that the sheath endpoint 12 and epiglottis cuff 6 are distal and inferior to the tip of the epiglottis. The endoscope 8 is then passed via the sheath 5 in step S704, providing a view of the vocal cords (S706), and the epiglottis cuff 6 inflated to optimize the view by elevating the epiglottis and dilating/splaying the laryngeal/hypopharyngeal tissues (S708). The endoscope 8 can be inserted whenever vocal cord motion needs to be assessed or left indwelling throughout the procedure.

In step S710, a recurrent laryngeal nerve candidate is identified as discussed herein. In step S712, the recurrent laryngeal nerve candidate is stimulated as discussed herein. The subject's vocal cords can be observed with endoscope 8 (S714b) and/or EMG electrodes (S714a) and the recurrently laryngeal nerve is identified (S716) as discussed herein.

A second method is displayed in FIGS. 2, 3, 4, and 7. In this method, the endoscope 8 is passed through the sheath 5 (S704) prior to insertion of the endotracheal tube 1 into the subject's larynx (S702). The endotracheal tube 1 is then passed through the vocal cords (S718), with or without a laryngoscope. During passage, the vocal cords are viewed using the laryngoscope or by viewing the vocal cords through the endoscope 8. In this technique, the endoscope tip may be secured to the tip of the endotracheal tube 1 with a fastener 10. In this technique, the endoscope 8 provides a view obtained from the tip of the tube 1. When tube 1 is passed inferior to the level of the vocal cords, the trachea is visualized. After passage of the endoscope 8 and endotracheal tube 1, the tube 1 is held in place and the endoscope 8 withdrawn to a level just above the vocal cords. The endoscope 8 can be left indwelling throughout the procedure or inserted whenever vocal cord motion needs to be assessed.

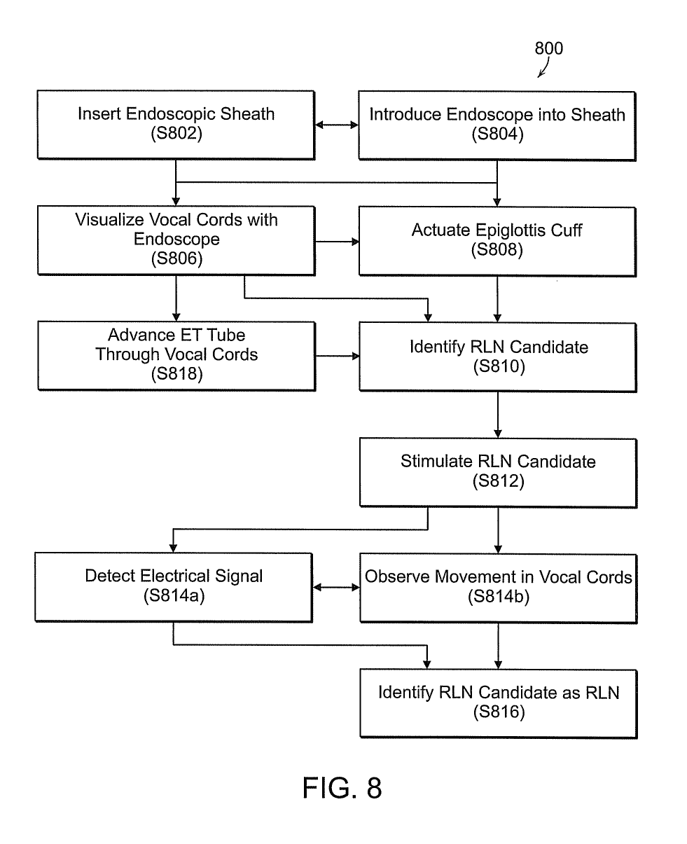

In another method 800 depicted in FIG. 8, the endoscopic conduit 60 can be used in other clinical situations to monitor the vocal cords in patients who have already been intubated. Endoscopic conduit 60 is designed to ride atop an endotracheal tube to the vocal cords and deliver the endoscope to that point. When using the endoscopic conduit 60, the anesthesiologist intubates the patient with a standard endotracheal tube or one of special design to accompany the endoscopic conduit. The endotracheal balloon cuff is inflated and the patient ventilated through it in a standard manner.

Shortly after intubation, or at anytime prior stimulation of the RLN, the endoscopic conduit 60 is passed by the anesthesiologist (S802). First, a standard laryngoscope can passed as was done during the intubation to elevate the epiglottis in the standard fashion. The distal end of the endoscopic conduit 60 can then be inserted into the patient's open mouth and the V-clip 66 is allowed to straddle the endotracheal tube. An endoscope 8 can be indwelling in the endoscopic conduit 60 sheath at this time or passed later (S804). The endoscopic conduit 60 can be advanced in a manner similar to an endotracheal tube, under direct visualization or visualization through the endoscope if it is indwelling. The V-clip 66 of the endoscopic conduit 66 will straddle the endotracheal tube, and keep the endoscopic conduit 60 atop the endotracheal tube, guiding it toward the vocal cords as it is passed.

The endoscopic conduit 60 is advanced until the epiglottic cuff 64 is in position such that when the cuff 64 is actuated the larynx is dilated and the epiglottis elevated. At this point the laryngoscope is removed, and the epiglottic cuff 64 is actuated (S808), holding the endoscopic conduit 66 in proper position and elevating the epiglottis out of the way. The endoscope 8 is passed via the sheath 63 if it is not already in position. The vocal cords will be visible (S806). The optional suction channel (not depicted) of the endoscopic conduit 60 can be used for instillation of fluids such as saline or defogging solution or evacuation of fluid and secretions. As with the combined endotracheal-tube-and-sheath devices described herein, the endoscopic conduit 60 can be attached to the patient and/or the endotracheal tube and/or the operating table to keep it in place during surgery (e.g., with tape). The endoscope 8 can be held in place in a similar manner. As with the endotracheal-tube-and-sheath devices described herein, the endoscope 8 can be left indwelling during surgery or passed just prior to testing and removed when it is no longer needed for monitoring. The endoscopic conduit 60 and indwelling endotracheal tube can then be used in steps S810-S818 in the manner discussed herein in the context of steps S710-S718.

In other embodiments, the endpoint 12 contains an attached camera and light source. In this embodiment, the combined endotracheal-tube-and-sheath devices could be passed and placed as described herein, but would not require passage of an endoscope 8 through the sheath as the camera is already pre-attached at end point 12.

In other embodiments of the endoscopic conduit 60, the endoscopic conduit 60 can be pre-attached to an endotracheal tube, in effect, creating the combined endotracheal-tube-and-sheath devices described herein. This device can then be passed as a single unit in a manner as described herein.

Embodiments of the invention are also useful in patients not requiring RLN monitoring. Some patients judged by the anesthesiologist to require an endotracheal tube 1 may have anatomy that precludes good visualization of the vocal cords during passage of a standard endotracheal tube. Such patients can be induced for anesthesia with a ventilation mask before an embodiment of the invention is used to provide visualization of the vocal cords during intubation. In this technique, the endoscope tip can be secured to the tip of the endotracheal tube 1 with a fastener 10. In this technique, the endoscope provides a view obtained from the tip of the tube 1. The tube 1 is passed through the lips and throat, giving a view of the vocal cords that could not be achieved using a standard laryngoscope. The tip of the endoscope 8 attached to the tip of the tube 1 with the fastener 10 is then passed inferior to the level of the vocal cords as is typically done during intubation with an endotracheal tube. When tube 1 is passed inferior to the level of the vocal cords, the trachea is visualized. After passage of the endoscope 8 and endotracheal tube 1, the tube 1 is held in place and the endoscope 8 withdrawn to a level just above the vocal cords if monitoring is needed, or altogether if it is not. In this manner, the device provides an option for passing an endotracheal tube in patients with challenging airway anatomy.

Referring to FIG. 5, a subject is undergoing neck surgery. Although the subject is depicted as a human, one of skill in the art will recognize that the apparatus and methods provided herein are equally applicable to other subjects such as mammals. A combined endotracheal-tube-and-sheath device is positioned in the subject's larynx. Endotracheal tube 1 is connected to anesthetic breath circuit for oxygenation, ventilation, and anesthesia.

An incision in the subject's neck allows for the removal of tissue, e.g. the thyroid gland. During the surgery, a recurrently laryngeal nerve candidate is identified. To evaluate whether the candidate is actually the RLN, an electric stimulation device 22 is applied to the RLN candidate. Suitable electrical stimulation devices are sold under the NEUROSIGN® trademark by The Magstim Company Limited of Carmarthenshire, Wales, United Kingdom. If the RLN candidate is actually the RLN, the subject's vocal cords will move as observed on monitor 18.

The foregoing specification and the drawings forming part hereof are illustrative in nature and demonstrate certain embodiments of the invention. It should be recognized and understood, however, that the description is not to be construed as limiting of the invention because many changes, modifications and variations may be made therein by those of skill in the art without departing from the essential scope, spirit or intention of the invention.

### EQUIVALENTS

Those skilled in the art will recognize, or be able to ascertain using no more than routine experimentation, many equivalents to the specific embodiments of the invention described herein. Such equivalents are intended to be encompassed by this invention.

### INCORPORATION BY REFERENCE

All publications, patent applications and patents identified herein are expressly incorporated herein by reference in their entirety.

1. An airway management device comprising:
an endotracheal tube having a proximal end and a distal end; and
a sheath adjacent to the endotracheal tube, the sheath having a proximal end and a distal end;
wherein the distal end of the endotracheal tube extends beyond the distal end of the sheath.

2. The airway management device of claim 1, further comprising a connector coupled to the endotracheal tube, the connector adapted to interface with an anesthetic breathing circuit.

3. The airway management device of claim 3, wherein the connector is removably coupled to the endotracheal tube or tethered to the endotracheal tube.

4. (canceled)

5. The airway management device of claim 1, wherein the endotracheal tube is flexible or rigid.

6. (canceled)

7. The airway management device of claim 1, wherein the endotracheal tube is armored.

8. The airway management device of claim 1, wherein the sheath is coupled to the endotracheal tube.

9. The airway management device of claim 8, wherein the sheath is coupled externally to the endotracheal tube or is coupled internally to the endotracheal tube.

10. (canceled)

11. The airway management device of claim 1, wherein the sheath is confluent with the endotracheal tube or is positioned on a concave (superior) side of the endotracheal tube.

12. (canceled)

13. The airway management device of claim 1, wherein the sheath includes one or more metal inserts to adjust and stiffen the endotracheal tube.

14. The airway management device of claim 1, wherein the sheath includes one or more channels for transportation of fluids.

15. The airway management device of claim 1, further comprising one or more cuffs coupled to the sheath.

16. The airway management device of claim 15 further comprising one or more inflation tubes coupled to the one or more cuffs.

17. The airway management device of claim 15, wherein the one or more cuffs are configured to dilate a subject's throat.

18. The airway management device of claim 15, wherein the distal end of the sheath extends beyond the one or more cuffs.

19. The airway management device of claim 18, wherein the distal end of the sheath extends between about 0 cm and about 10 cm beyond the one or more cuffs.

20. The airway management device of claim 1, wherein the sheath has an internal diameter selected from the group consisting of: less than about 2 mm, between about 2 mm and about 10 mm, and greater than about 10 mm.

21. The airway management device of claim 1, wherein the distal end of the sheath is configured to provide a view of a subject's vocal cords when the airway management device is placed in the larynx of the subject.

22. The airway management device of claim 1 further comprising a lens coupled to an opening at the distal end of the sheath.

23. The airway management device of claim 1 further comprising a prism coupled to an opening at the distal end of the sheath.

24. The airway management device of claim 1 further comprising a cap coupled to the an opening at the proximal end of the sheath.

25. The airway management device of claim 24 wherein the cap is tethered to the sheath.

26. The airway management device of claim 1 further comprising a flap coupled to the proximal opening of the sheath.

27. The airway management device of claim 1 further comprising a flap coupled to the distal opening of the sheath.

28. The airway management device of claim 1, wherein the sheath is configured to receive an endoscope.

29. The airway management device of claim 28, further comprising an endoscope having a proximal end and a distal end.

30. The airway management device of claim 28, further comprising a connector coupled to the distal end of the endotracheal tube and configured to immobilize the endoscope.

31. The airway management device of claim 28 wherein the endoscope includes a light source.

32. The airway management device of claim 28 wherein the endoscope includes a camera located at the distal end of the endoscope.

33. The airway management device of claim 28 wherein the endoscope includes an ocular located at the proximal end of the endoscope.

34. The airway management device of claim 28 wherein the endoscope includes a first fiber optic cable for transmission of light from the proximal end of the endoscope to the distal end of the endoscope.

35. The airway management device of claim 34 wherein the endoscope includes a second fiber optic cable for transmission of images from the distal end of the endoscope to the proximal end of the endoscope.

36. The airway management device of claim 28 wherein the endoscope includes one or more channels for transportation of fluids.

37. The airway management device of claim 28 wherein the endoscope is rigid, semi-rigid or flexible.

38. (canceled)

39. (canceled)

40. The airway management device of claim 28 wherein the endoscope is steerable.

41. The airway management device of claim 1, further comprising an additional cuff coupled to the endotracheal tube.

42. A surgical kit comprising an airway management device of claim 1 and instructions for use.

43-45. (canceled)

46. A method of identifying a subject's recurrent laryngeal nerve, the method comprising:
introducing an airway management device of claim 1 into the subject's larynx;
introducing an endoscope into the sheath;
visualizing the subject's vocal cords with the endoscope;
identifying a recurrent laryngeal nerve candidate;
stimulating the recurrent laryngeal nerve candidate;
observing a movement in the subject's vocal cords with the endoscope; and
identifying the recurrent laryngeal nerve candidate as the subject's recurrent laryngeal nerve.

47. A method of protecting a subject's recurrent laryngeal nerve during surgery, wherein the subject was previously intubated with an endotracheal tube, the method comprising:
introducing an airway management device of claim 1 into the subject's larynx;
introducing an endoscope into the sheath;
visualizing the subject's vocal cords with the endoscope;
identifying a recurrent laryngeal nerve candidate;
stimulating the recurrent laryngeal nerve candidate;
observing a movement in the subject's vocal cords with the endoscope; and
identifying the recurrent laryngeal nerve candidate as the subject's recurrent laryngeal nerve,
thereby protecting the subject's recurrent laryngeal nerve during surgery.

48-51. (canceled)

52. An endoscopic conduit comprising:
a sheath having a proximal end and a distal end; and
a clip coupled to the distal end of the sheath, the clip configured to straddle an endotracheal tube.

53-61. (canceled)

62. An airway management device comprising:
an endotracheal tube; and
an endoscopic conduit of claim 52.

63. (canceled)

64. (canceled)

65. An airway management device comprising:
an endotracheal tube having a proximal end and a distal end, the endotracheal tube having at least one portion with a non-circular cross-section; and
a sheath adjacent to the endotracheal tube, the sheath having a proximal end and a distal end;
wherein the distal end of the endotracheal tube extends beyond the distal end of the sheath.

66-73. (canceled)

74. An airway management device comprising:
an endotracheal tube having a proximal end and a distal end; and
a sheath adjacent to the endotracheal tube, the sheath having a proximal end and a distal end, wherein the sheath includes a camera at the distal end;
wherein the distal end of the endotracheal tube extends beyond the distal end of the sheath.

75-83. (canceled)

84. An airway management device comprising:
an endotracheal tube having a proximal end and a distal end;
a sheath adjacent to the endotracheal tube; and
a foam cuff coupled to the sheath.

85-88. (canceled)

89. An endoscopic conduit comprising:
a sheath having a proximal end and a distal end;
a V-clip coupled to the distal end of the sheath, the V-clip configured to straddle an endotracheal tube; and
one or more attachment devices for attachment to an endotracheal tube.

90-94. (canceled)

95. An airway management device comprising:
an endotracheal tube;
a sheath adjacent to the endotracheal tube; and
one or more EMG electrodes.

96-101. (canceled)

102. An airway management device comprising:
an endotracheal tube; and
one or more visualization devices coupled to the endotracheal tube.

103-109. (canceled)

110. An endoscope comprising:
a visualization device; and
a V-clip coupled to the visualization device.

111-113. (canceled)
